(19) United States
(12) Patent Application Publication
(10) Pub. No.: US 2007/0270880 A1
(43) Pub. Date: Nov. 22, 2007

(54) Bone screw revision tools and methods of use

(76) Inventors: Gary S. Lindemann, Collierville, TN (US); Joe W. Ferguson, Collierville, TN (US); Marc Paul, Memphis, TN (US)

Correspondence Address: COATS & BENNETT, PLLC, 1400 Crescent Green, Suite 300, Cary, NC, 27518, US

(21) Appl. No.: 11/413,279

(22) Filed: Apr. 28, 2006

(51) Int. Cl. A61F 2/00

(52) U.S. Cl. 606/104

(57) ABSTRACT

The present application is directed to tools that may generally deflect a locking ring away from the fastener, and also engage a head of the fastener to rotate and remove the fastener from a vertebral member. The tools may include a first member that contacts and moves the locking ring away from the aperture 101. A second member engages the head and transfers torque to the fastener for removal from the vertebral member.

### BACKGROUND

The human spine is a biomechanical structure consisting of thirty-three vertebral members and is responsible for protecting the spinal cord, nerve roots and internal organs of the thorax and abdomen. The spine also provides structural support for the body while permitting flexibility of motion. In certain surgical procedures it is necessary to secure together two or more of the vertebral members. The procedure may be necessary for example as a result of physical trauma or degenerative diseases.

One type of surgical procedure includes attachment of a vertebral plate to the vertebral members. The vertebral plate is sized to extend across two or more of the vertebral members. One or more bone screws extend through apertures in the plate and into the vertebral members to secure the plate. One issue with the attachment is that the screws may loosen and back-out of the vertebral members. Screw retention devices may be necessary to prevent the screw from backing-out of the vertebral members.

In some instances, it becomes necessary or desirable to remove the vertebral plate. To do so, the screw retention devices may need to be disengaged prior to removal of the screws. Additionally, the screw holes in the vertebral members may have stripped, making the removal of screws therefrom difficult. Improvements are desired.

### SUMMARY

The present application is directed to tools, apparatus, systems and methods for revising or removing screws from a bone. The present application will be particularly useful for removing screws from a vertebral body, such as screws used to hold an anterior cervical plate to a patient's spine. The apparatus and methods also will be useful for removing screws that are stripped in the bone.

In one embodiment of an orthopedic instrument for revising a screw that couples a bone plate to a bone, the instrument includes an elongate member having a handle at a proximal end and a screw engagement portion at a distal end. The screw engagement portion is adapted to expand to engage a screw head. The instrument includes a sleeve coupled to the elongate member. The sleeve is adapted to deflect a screw retention device. In this manner, the instrument helps deflect a screw retention device, while also engaging a screw to be removed.

In one aspect, the screw engagement portion includes first and second tip portions having a hexagonal shape when in a first position. The first and second tip portions are spaced apart when the tip portions are in a second position. In one aspect, the instrument includes a tension device adapted to bias the first and second tip portions towards the second position. The instrument may further include a lever, handle, grip, or the like, coupled to the first and second tip portions, for transferring the tip portions to the first position.

In one aspect, the screw engagement portion includes first and second tip portions having a circumferential ridge. In alternative embodiments, the circumferential ridge is adapted to engage a recess in the screw head, is adapted to engage a circumferential groove in the screw head recess, or is adapted to frictionally engage the screw head recess.

In some aspects, the sleeve is rotatably coupled to the elongate member. The sleeve also may be removably coupled to the elongate member. In some aspects, the sleeve includes a distal tip extending around at least a portion of a sleeve circumference. The sleeve may be adapted to rotate relative to the screw engagement portion, with the distal tip adapted to deflect the screw retention device. In some aspects, the sleeve is axially translatable relative to the screw engagement portion to translate the distal tip beyond a distal end of the screw engagement portion.

In one aspect, the sleeve includes a distal end having outer and inner edges. The outer edge is adapted to engage the screw retention device and the inner edge is adapted to engage the screw head. In some embodiments, the inner edge is an undercut inner edge, and/or includes a lip adapted to engage the screw head.

The present application provides tools for removing a screw from an orthopedic plate of the type having at least one aperture therein for receiving the screw and a screw retaining member engaging a head of the screw. In one embodiment, the tool includes a handle and an elongate sleeve member rotatably coupled to the handle. The sleeve member has a proximal end and a distal end, with the sleeve member distal end having an outer edge and an inner edge. The sleeve member outer edge is adapted to translate the screw retaining member away from the screw head when the sleeve is rotated relative to the handle. The sleeve inner edge is adapted to grip an outer surface of the screw head. In some aspects, the sleeve inner edge includes a foot member and/or an angled edge that is adapted to engage a groove in the screw head.

In still another embodiment, the tool for removing the screw includes a first elongate member rotatably coupled to a second elongate member in a generally coaxial configuration. The second elongate member includes a distal tip for engaging a tool-receiving recess in a screw head. The first elongate member is adapted to extend outside a periphery of the screw head to engage the screw retaining member.

In one aspect, the distal tip of the second elongate member includes a left-hand threaded tip. Alternatively, the distal tip of the second elongate member includes a split hex tip. In a particular aspect, the first member further includes a notch adapted to receive an edge of the screw retaining member. The first member is adapted to translate the screw retaining member away from the screw head upon rotation of the notch away from the screw retaining member.

The present invention further provides methods for removing a screw from a bone. In one embodiment, the method includes providing a screw revision tool in accordance with any of the embodiments described herein. For example, the tool may include an elongate member having a handle at a proximal end and a screw engager at the distal end, and a sleeve coupled to the elongate member. One method includes translating the screw retention device away from the screw head with the sleeve, gripping the screw head with the screw engager, and axially translating the screw with the screw engager while the sleeve maintains the screw retention device away from the screw head. In this manner, the screw retention device is positioned to be out of the path of the screw to be removed, using a same tool for both processes.

In one aspect, translating the screw retention device includes rotating the sleeve. In another aspect, gripping the screw head includes engaging a recess in the screw head with the screw engager. The screw engager may include a split hex tip, and the method may further include inserting the split hex tip into the recess when the split hex tip is in a first position, and expanding the split hex tip to a second position to engage the recess. This method will be particularly useful for engaging screws that are or may be stripped in the bone.

In another aspect, the screw engager includes a left-hand threaded tip. In this aspect, gripping the screw head includes screwing the threaded tip into the screw head. In one aspect, screwing the screw engager threaded tip into the screw head creates a threaded engagement between the screw head and tip. This may occur, in some embodiments, when the screw engager threaded tip is a self drilling threaded tip.

In one aspect, gripping the screw head includes engaging a periphery of the screw head with the screw engager. This occurs, in one aspect, when the screw engager includes a lip to engage the screw head periphery.

Other features and advantages of the invention will appear from the following description in which the preferred embodiment has been set forth in detail in conjunction with the accompanying drawings.

### BRIEF DESCRIPTION OF THE DRAWINGS

FIG. 1 is a top view illustrating a vertebral plate with a locking ring that contains a fastener according to one embodiment.

FIG. 2 is a perspective view illustrating a tool according to one embodiment.

FIG. 3 is an exploded side view illustrating a tool according to one embodiment.

FIG. 4 is a perspective view illustrating a sleeve according to one embodiment.

FIG. 5 is a cross section view illustrating a tool engaged with a locking ring and a fastener according to one embodiment.

FIG. 6 is a cross section view illustrating a tool engaged with a locking ring and a fastener according to one embodiment.

FIG. 7 is a cross section view illustrating a tool engaged with a locking ring and a fastener according to one embodiment.

FIG. 8 is a partial perspective view illustrating an intermediate member according to one embodiment.

FIG. 9 is a side view illustrating a tool according to one embodiment.

FIG. 10 is a side view illustrating a tool according to one embodiment.

### DETAILED DESCRIPTION

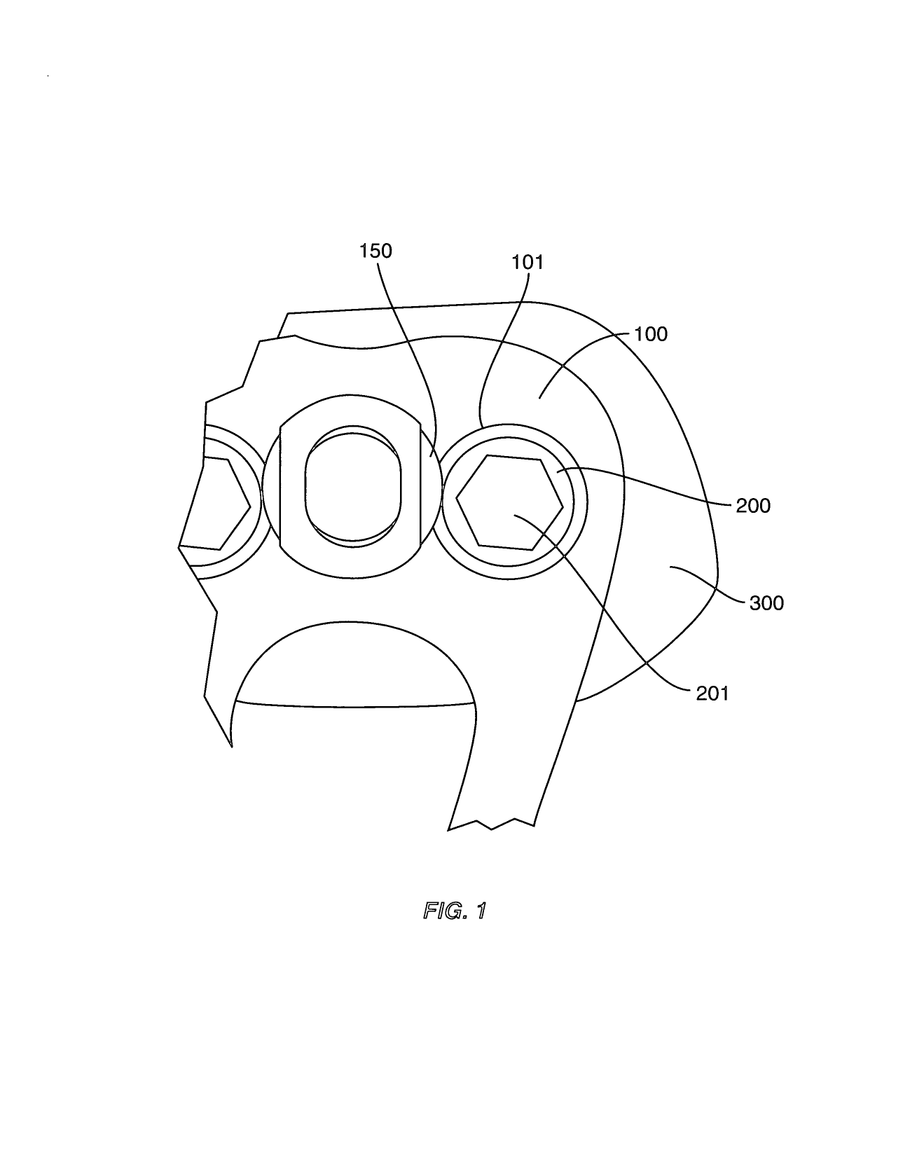

The present invention is directed to revision tools, apparatus, systems and methods for removing one or more fasteners from an orthopedic implant, such as a vertebral plate. FIG. 1 illustrates one embodiment of a vertebral plate 100 including an aperture 101. A fastener 200 is mounted within the aperture 101 to mount the plate 100 to a vertebral member 300. The plate 100 includes a locking ring 150 that extends over the fastener 200 to prevent back-out from the vertebral member 300. Removal of the fastener 200 during a revision procedure requires that the locking ring 150 is deflected away from the fastener 200. After deflection, the fastener 200 is engaged and rotated to remove the fastener 200. Examples of vertebral plates with locking rings include the VENTURE™ Anterior Cervical Plate System available from Medtronic Sofamor Danek of Memphis, Tenn., and U.S. patent application Ser. No. 10/870,026 filed Jun. 17, 2004 and entitled “Method and Apparatus for Retaining Screws in a Plate”, each of which is incorporated herein by reference.

The tools disclosed herein generally deflect the locking ring 150 away from the fastener 200, and also engage a head 201 of the fastener 200 to rotate and remove the fastener 200 from the vertebral member 300. The tools may include a first member that contacts and moves the locking ring 150 away from the aperture 101. A second member engages the head 201 and transfers torque to the fastener 200 for removal from the vertebral member 300.

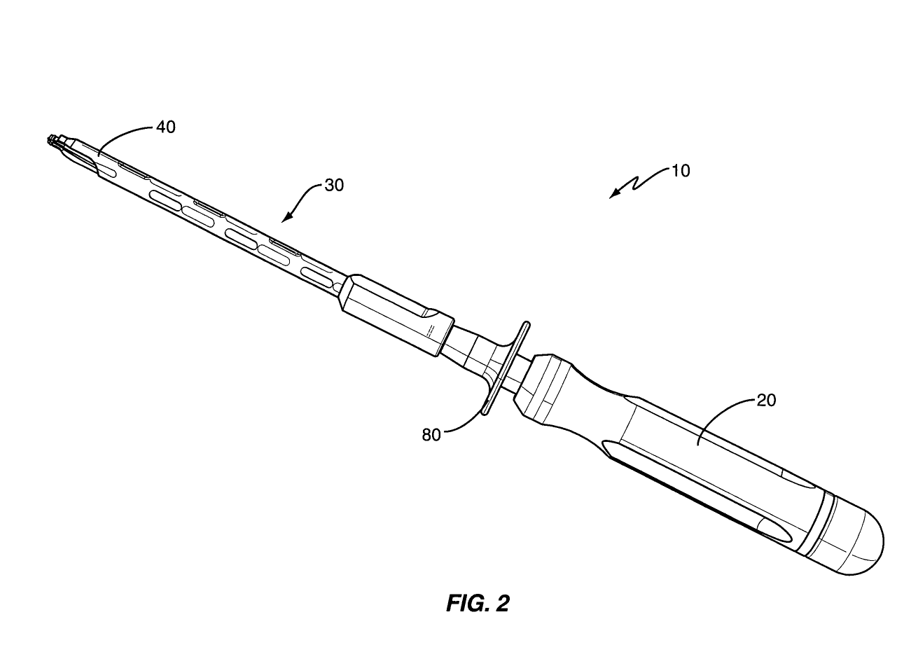

FIG. 2 illustrates a tool 10 according to one embodiment. Tool 10 includes a handle 20 for grasping by the surgeon, and a shaft 30 that deflects the locking ring 150 and engages the fastener 200. The tool 10 may be substantially straight as illustrated in FIG. 2, or one or both of the handle 20 and shaft 30 may be curved. The overall length of the shaft 30 may allow the handle 20 to remain on the exterior of a patient while the distal end of the shaft 30 is engaged with the fastener 200.

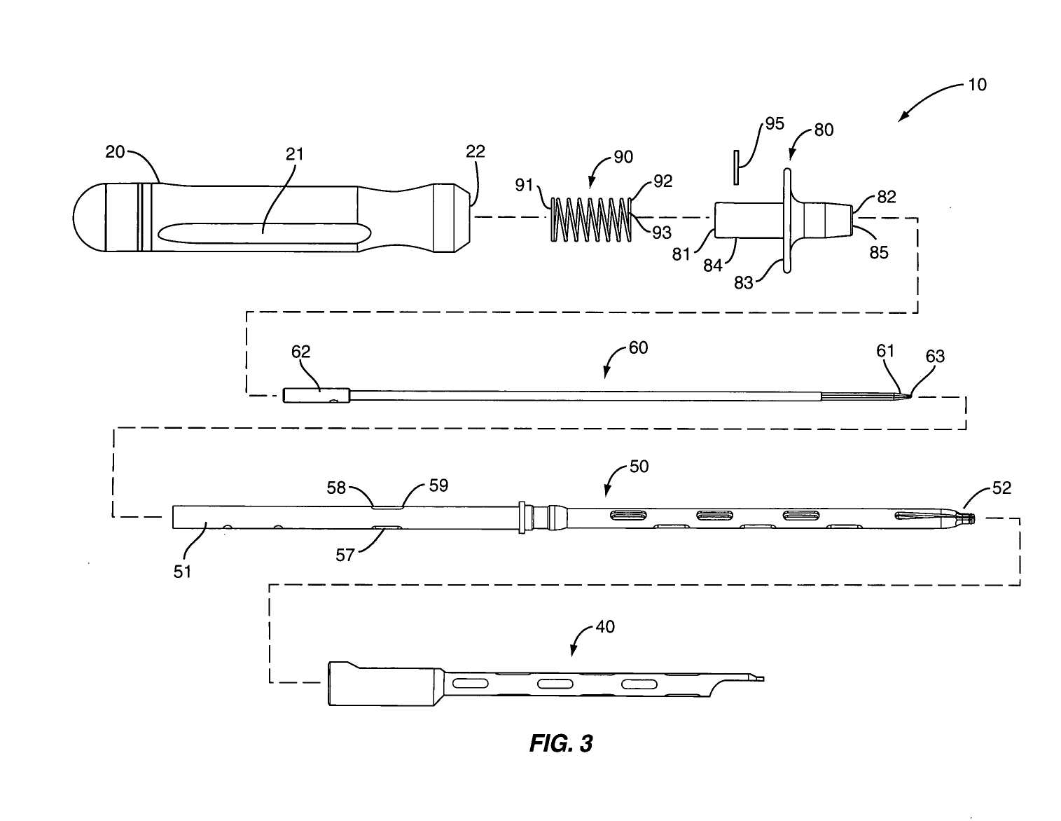

FIG. 3 illustrates an exploded view of the tool 10. The shaft 30 comprises a sleeve 40, an intermediate member 50, and a rod 60. These elements extend outward from the handle 20 in an overlapping configuration. In one embodiment, sleeve 40, intermediate member 50 and rod 60 are coaxially aligned. The rod 60 is positioned within the interior and is overlapped by the intermediate member 50, which is overlapped by the sleeve 40.

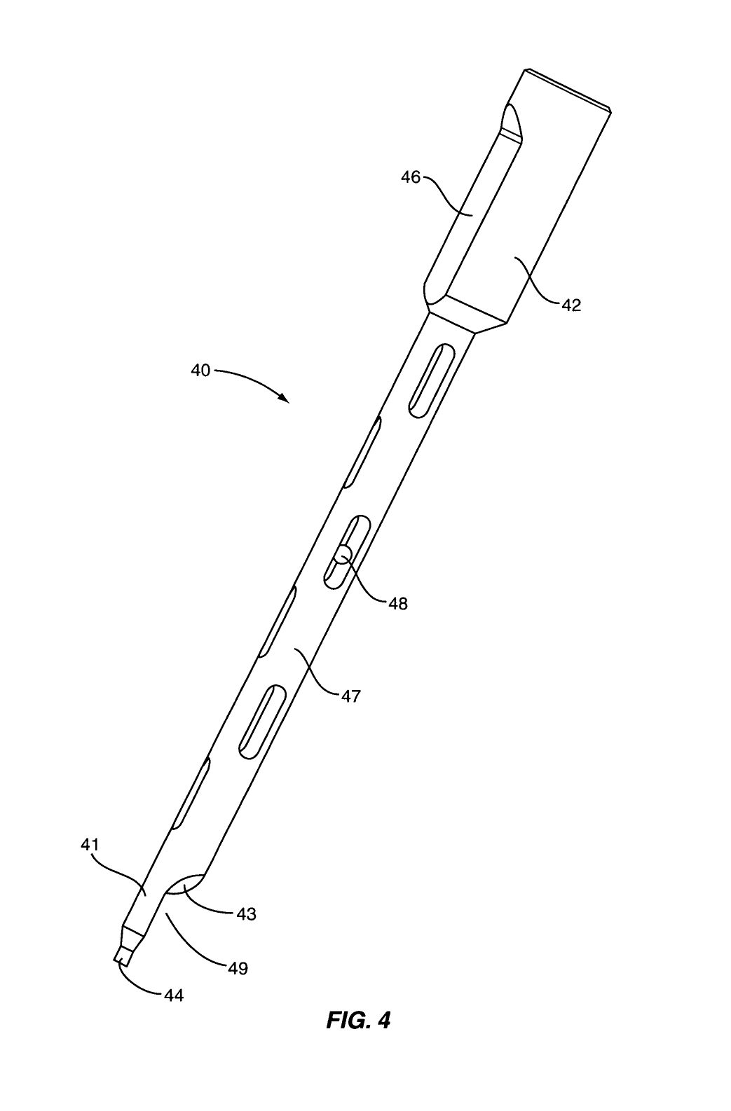

Sleeve 40 contacts and deflects the locking ring 150 away from the aperture 101 and inserted fastener 200. FIG. 4 illustrates an embodiment of the sleeve 40 that includes a distal section 41, intermediate section 47, and a proximal section 42. The intermediate section 47 and proximal section 42 are substantially enclosed to form an interior 43 sized to extend around the intermediate member 50. The proximal section 42 may include a gripping surface 46 to rotate and/or remove the sleeve 40 from the intermediate member 50. Gripping surface 46 may be substantially flat, and may include a surface finish to prevent slipping. Gripping surface 46 also provides a feature that can be tactilely felt by the surgeon during the procedure. In some instances, the outer sleeve 40 is positioned within the wound and is not visible. The surgeon may feel the gripping surface 46 to manipulate the outer sleeve 40 as necessary.

Attachment members 48, such as one or more ball seals, may be positioned within the interior 43 to contact the intermediate member 50 and maintain attachment of the sleeve 40. Alternatively, attachment members 48 may be positioned on the intermediate member 50 to maintain the attachment. Various attachment members 48 may be used for attachment including threaded connections, ball-and-detent configuration, snap ring, and general interference fits. In some embodiments, sleeve 40 is removably attached to intermediate member 50. In this manner, sleeve 40 may be removed for cleaning, replacement, and the like. In some embodiments, sleeve 40 is coupled to intermediate member 50 in a manner which permits relative rotation between the two components as further described below.

The distal section 41 extends outward from an intermediate section 47. Unlike the intermediate section that forms an enclosure around the interior 43, the distal section 41 includes an open side 49. A single distal section 41 may extend outward from the intermediate section 47 as illustrated in FIG. 4, or two or more distal sections 41 may extend outward. The distal section 41 includes a tip 44 sized to contact and deflect the locking ring 150. The tip 44 may be curved as illustrated in FIG. 4, or may be substantially straight.

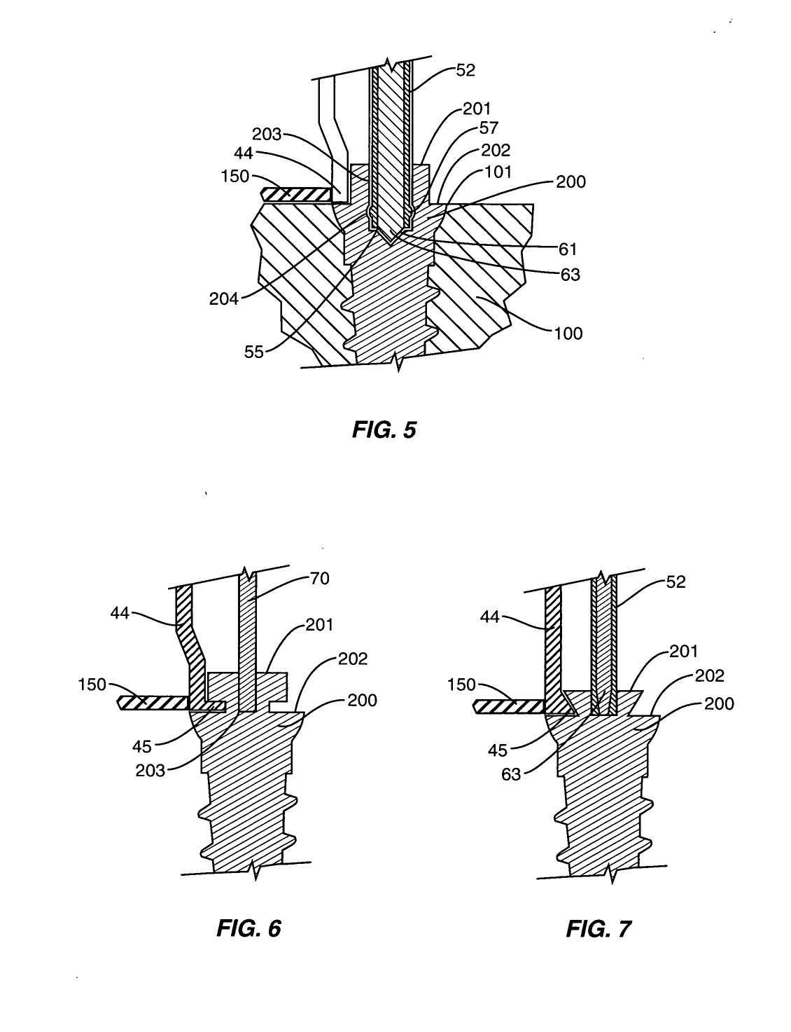

FIG. 5 illustrates the tip 44 contacting and deflecting the locking ring 150 away from the aperture 101 and fastener 200. In this embodiment, the tip 44 is inserted to contact the head 201 of the fastener 200. Initially, the distal section 41 is aligned with the opening side 49 over the locking ring 150 and the tip 44 positioned against a shelf 202 on the head 201. The sleeve 40 is then rotated causing the tip 44 to slide along the shelf 202 and contact and deflect the locking member 150 away from the aperture 101 and fastener 200. In this embodiment, the tip 44 includes a relatively constant thickness. FIG. 6 illustrates another embodiment with the tip 44 including a leg 45 that extends outward from the distal end. In use, the tip 44 contacts and deflects the locking ring 150, and the leg 45 fits within a groove in the fastener head 201 to attach the sleeve 40 to the fastener 200. In the embodiment of FIG. 6, the fastener head 201 includes a slot or undercut edge (not illustrated) to allow the leg 45 to be inserted beyond the head 201 to abut against the shelf 202. FIG. 7 illustrates a similar embodiment with the tip 44 being flared to fit within a groove or angled edge in the head 201.

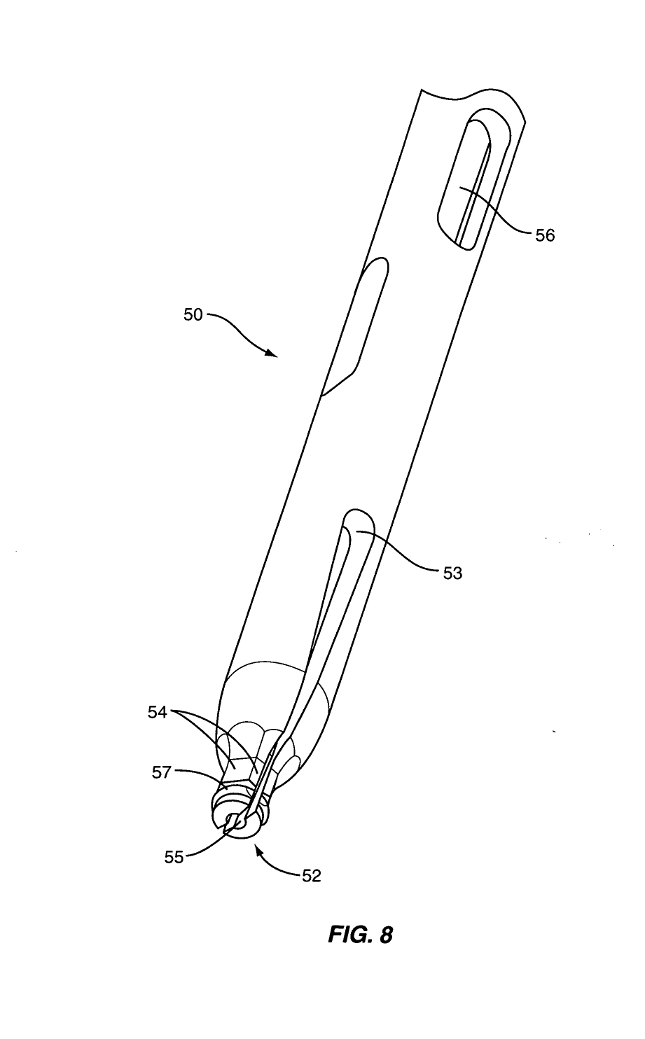

The intermediate section 50 and the rod 60 may work in combination to engage the fastener 200. The intermediate member 50 is positioned within the sleeve 40 and includes a slotted tip 52 and a proximal end 51. FIG. 8 illustrates the distal section of the intermediate member 50. The tip 52 is positioned at the distal end and includes one of more inwardly-extending slots 53. The slots 53 allow for the width of tip 52 to expand and contract as will be explained below. The number of slots 53 may vary depending upon the context, and the lengths and widths of the slots 53 may also vary. Engagement surfaces 54 are positioned at the tip 52 to contact a receptacle 203 in the fastener head 201. In one embodiment, six engagement surfaces 54 are positioned at the tip 52 to form a hexagonal shape that engages a hexagonal fastener head 201. The tip 52 may also include other configurations, including Torx, Quad, and other head types. Each of the surfaces 54 may include the same shape and size, or the surfaces 54 may be different. In a particular embodiment, tip 52 is a split-hex shape, with tip 52 bifurcated by slot 53. In this embodiment, each half of tip 52 includes three engagement surfaces 54. Other combinations of surfaces 54 and slots 53 also fall within the scope of the present invention. In some embodiments, an extension 57 extends outward from one or more of the surfaces 54 to engage with fastener receptacle 203. In a particular embodiment, extension 57 comprises a raised ring or lip which engages detent(s) 204 in the fastener receptacle 203. The tip 52 may also include an opening 55 that extends into a hollow interior 56 of the intermediate member 50.

The proximal section 51 as illustrated in FIG. 3 is configured to mount to the handle 20. The proximal section 51 may be fixedly connected to the handle 20 by an adhesive, mechanical fastener, or other methods known in the art. A slot 57 is positioned through the proximal section 51 at a point distal from the connection with the handle 20. Slot 57 extends axially along a length of the outer walls and includes a first edge 58 and a second edge 59. The slot 57 is sized to receive a pin 95 as will be explained in detail below.

The rod 60 is positioned within the intermediate section 50. Rod 60 includes a tapered distal end 61 and a proximal end 62. The tapered end 61 includes a tip 63 and tapers inward a predetermined distance. The length of the taper may vary depending upon the application.

A grip 80 is attached to the proximal end 62 of the rod 60. Grip 80 includes a first end 81, second end 82, and a flange 83. The first end 81 faces the handle 20 and includes a contact surface for a biasing member 90. In one embodiment, the first end 81 includes a receptacle with outer sidewalls to receive the biasing member 90. The second end 82 connects with the proximal end 62 of the rod 60. The second end 82 may include a receptacle with outer sidewalls to receive the proximal end 62 of the rod 60, or provide a surface against which the end 62 abuts. The flange 83 includes an enlarged width for grasping by the surgeon as will be explained in detail below.

Grip 80 further includes an aperture 84 sized to receive the pin 95. Pin 95 further extends through the slot 57 in the intermediate section 50. This connection provides for the grip 80 and attached rod 60 to axially move relative to the intermediate member 50. The extent of axial movement is dependent upon the length of the slot 57 defined between edges 58, 59, and a thickness of the pin 95. The movement of the grip 80 is on the exterior of the intermediate member 50 with the rod 60 moving on the interior.

The handle 20 provides a surface for the surgeon or other user to grasp and manipulate the tool 10. Surface features 21 may be positioned along the handle 20 to facilitate these efforts. The surface features 21 may include finger grips, indents, knurled finish, etc. The handle 20 of FIG. 2 is substantially straight, although other shapes and configurations are also considered. The distal end 22 contacts the biasing member 90. The distal end 22 may include a receptacle with outer sidewalls to receive the member 90.

The biasing member 90 is positioned between the handle 20 and the grip 80. Biasing member 90 includes a first end 91 in contact with the handle 20 and a second end 92 in contact with the grip 80. The biasing member 90 biases the grip 80 and attached rod 60 in a distal direction relative to the handle 20 and intermediate section 50. In one embodiment, biasing member 90 is a coil spring with a central section 93 sized to receive the intermediate section 50. Various different biasing members 90 may also be utilized as is known in the art.

In use, the grip 80 and attached rod 60 are moved in a proximal direction relative to the intermediate member 50. This causes the tip 52 of the intermediate section 50 to have a reduced sized. The tip 52 is then placed within the receptacle 203 in the fastener head 201. The grip 80 is then released allowing the tapered end 61 of the rod 60 to axially move through the interior 56 of the intermediate member 50. This axial movement, in one embodiment, occurs as a result of biasing member 90 pressing against grip 80. As illustrated in FIG. 5, the tip 63 moves through the opening 55 in the intermediate section tip 52. This causes the tip 52 to move radially outward or expand and engage the surfaces of the fastener receptacle 203. The expansion of tip 52 occurs, in part, due to the engagement between the inner surface of tip 52 and the outer surface of tapered end 61. The intermediate section 50 may then be rotated to remove the fastener 200 from the vertebral member 300. In this manner, the engagement of tip 52 and fastener 200 allows for fastener 200 to be removed from the vertebral member 300 even when the fastener 200 is stripped therein.

In the embodiment of FIG. 5, the rod tip 63 extends outward beyond the intermediate member tip 52. In other embodiments as illustrated in FIG. 7, the rod tip 63 remains within the intermediate member 52. As further illustrated in FIG. 5, extensions 57 may mount within detents 204 in the fastener receptacle 203 to further engage the fastener 200.

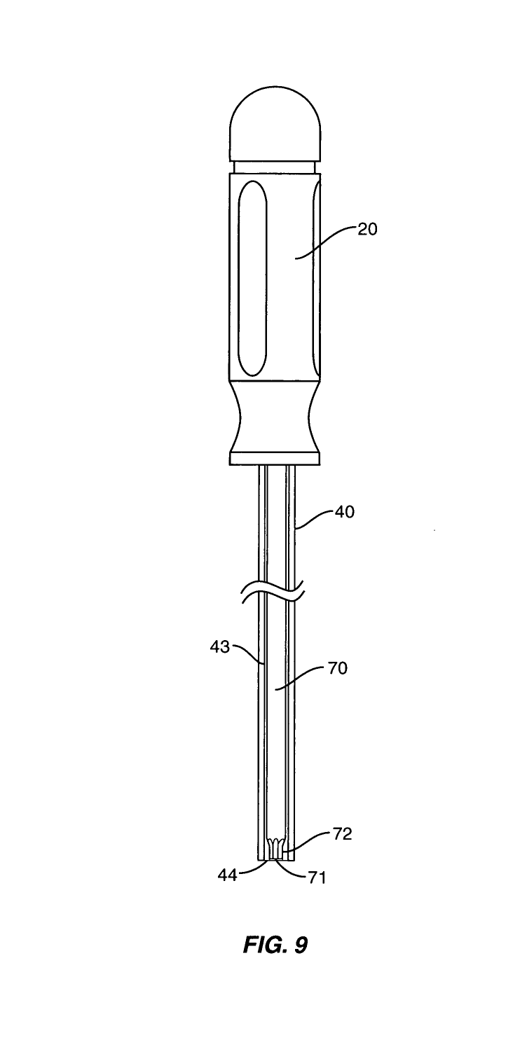

The embodiment described above includes the intermediate section 50 and the rod 60 working in combination to engage the fastener 200. FIG. 9 illustrates another embodiment with a shaft 70 extending through the interior 43 of the sleeve 40. The shaft 70 includes a tip 71 with engagement surfaces 72 that contact the fastener receptacle 203. The number of engagement surfaces 72 matches the shape of the receptacle 203. Engagement surfaces 72 may be substantially flat, or may be shaped such as Torx, Quad, and other screw head types. The shaft 70 may be fixedly positioned within the sleeve 40, or may be axially extendable. In use, the sleeve 40 and shaft 70 are positioned over the plate 100. The sleeve 40 is positioned adjacent to the locking ring 150 and the tip 71 is inserted within the receptacle 203. The sleeve 40 is rotated relative to the shaft 70 to deflect the locking ring 150 away from the fastener 200. Once deflected, the shaft 70 is rotated to remove the fastener 200.

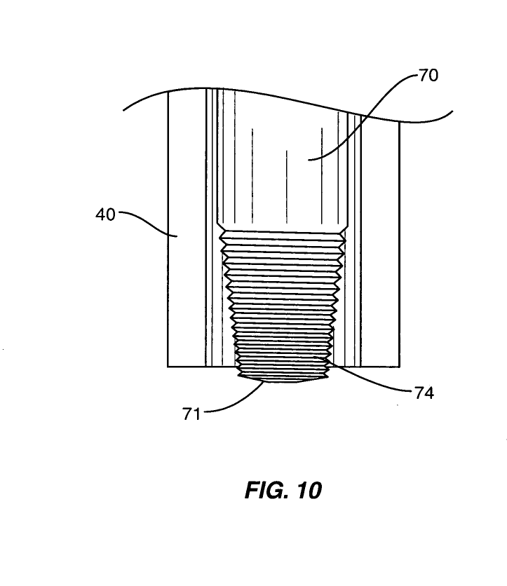

FIG. 10 illustrates another embodiment with the shaft 70 including a threaded tip 71. The threads 71 may bite into the fastener 200 for removal from the vertebral member 300. In one embodiment, the threads 71 are reversed such that rotation of the shaft 70 in a first direction causes the threads 71 to bite into and attach with the fastener 200. In one embodiment, threads 71 comprise a left-hand threaded region. Once threads 71 and fastener 200 are engaged or otherwise attached, rotation of the shaft 70 in a second direction removes the fastener 200 from the vertebral member 300. Engaging the threaded tip 71 into the fastener 200 may cause deformation of the receptacle 203. In another embodiment, the distal tip includes flutes that bite into and engage the fastener 200.

The term “distal” is generally defined as in the direction of the patient, or away from a user of a device. Conversely, “proximal” generally means away from the patient, or toward the user. Spatially relative terms such as “under”, “below”, “lower”, “over”, “upper”, and the like, are used for ease of description to explain the positioning of one element relative to a second element. These terms are intended to encompass different orientations of the device in addition to different orientations than those depicted in the figures. Further, terms such as “first”, “second”, and the like, are also used to describe various elements, regions, sections, etc and are also not intended to be limiting. Like terms refer to like elements throughout the description.

As used herein, the terms “having”, “containing”, “including”, “comprising” and the like are open ended terms that indicate the presence of stated elements or features, but do not preclude additional elements or features. The articles “a”, “an” and “the” are intended to include the plural as well as the singular, unless the context clearly indicates otherwise.

The present invention may be carried out in other specific ways than those herein set forth without departing from the scope and essential characteristics of the invention. The present embodiments are, therefore, to be considered in all respects as illustrative and not restrictive, and all changes coming within the meaning and equivalency range of the appended claims are intended to be embraced therein.

What is claimed is:

1. An orthopedic instrument for revising a screw coupling a bone plate to a bone, the instrument comprising:
an elongate member having a handle at a proximal end and a screw engagement portion at a distal end, the screw engagement portion adapted to expand to engage a screw head; and
a sleeve coupled to the elongate member;
wherein the sleeve is adapted to deflect a screw retention device.

2. The instrument as in claim 1 wherein the screw engagement portion comprises first and second tip portions having a hexagonal shape when in a first position.

3. The instrument as in claim 2 wherein the first and second tip portions are spaced apart when the tip portions are in a second position.

4. The instrument as in claim 3 comprising a tension device adapted to bias the first and second tip portions towards the second position.

5. The instrument as in claim 3 further comprising a lever coupled to the first and second tip portions for transferring the tip portions to the first position.

6. The instrument as in claim 1 wherein the screw engagement portion comprises first and second tip portions having a circumferential ridge.

7. The instrument as in claim 6 wherein the circumferential ridge is adapted to engage a recess in the screw head.

8. The instrument as in claim 7 wherein the circumferential ridge is adapted to engage a circumferential groove in the screw head recess.

9. The instrument as in claim 7 wherein the circumferential ridge is adapted to frictionally engage the screw head recess.

10. The instrument as in claim 1 wherein the sleeve is rotatably coupled to the elongate member.

11. The instrument as in claim 1 wherein the sleeve is removably coupled to the elongate member.

12. The instrument as in claim 1 wherein the sleeve comprises a distal tip extending around at least a portion of a sleeve circumference.

13. The instrument as in claim 12 wherein the sleeve is adapted to rotate relative to the screw engagement portion, the distal tip adapted to deflect the screw retention device.

14. The instrument as in claim 12 wherein the sleeve is axially translatable relative to the screw engagement portion to translate the distal tip beyond a distal end of the screw engagement portion.

15. The instrument as in claim 1 wherein the sleeve comprises a distal end having outer and inner edges, the outer edge being adapted to engage the screw retention device and the inner edge being adapted to engage the screw head.

16. The instrument as in claim 15 wherein the inner edge is an undercut inner edge.

17. The instrument as in claim 15 wherein the inner edge comprises a lip adapted to engage the screw head.

18. A tool for removing a screw from an orthopedic plate of the type having at least one aperture therein for receiving the screw, the plate having a screw retaining member engaging a head of the screw, the tool comprising:
a handle;
an elongate sleeve member rotatably coupled to the handle, the sleeve member having a proximal end and a distal end, the sleeve member distal end having an outer edge and an inner edge;
wherein the sleeve member outer edge is adapted to translate the screw retaining member away from the screw head when the sleeve is rotated relative to the handle; and
wherein the sleeve inner edge is adapted to grip an outer surface of the screw head.

19. The tool as in claim 18 wherein the sleeve inner edge comprises a foot member adapted to engage a groove in the screw head.

20. The tool as in claim 18 wherein the sleeve inner edge comprises an angled edge adapted to engage a groove in the screw head.

21. A tool for removing a screw from an orthopedic plate of the type having at least one aperture therein for receiving the screw, the plate having a screw retaining member engaging a head of the screw, the tool comprising:
a first elongate member rotatably coupled to a second elongate member in a generally coaxial configuration;
the second elongate member comprising a distal tip for engaging a tool-receiving recess in the screw head; and
wherein the first elongate member is adapted to extend outside a periphery of the screw head to engage the screw retaining member.

22. The apparatus as in claim 21 wherein the distal tip of the second elongate member comprises a left-hand threaded tip.

23. The apparatus as in claim 21 wherein the distal tip of the second elongate member comprises flutes that tap into a head of the screw.

24. The apparatus as in claim 21 wherein the distal tip of the second elongate member comprises a split hex tip.

25. The apparatus as in claim 21 wherein the first member further comprises a notch adapted to receive an edge of the screw retaining member, the first member adapted to translate the screw retaining member away from the screw head upon rotation of the notch away from the screw retaining member.

26. The instrument as in claim 21 wherein the first elongated member is removably coupled to the second elongate member.

27. A method of removing a screw from an orthopedic plate, the plate having a screw retention device engaging a portion of a head of the screw, the method comprising:
providing a screw revision tool comprising:
an elongate member having a handle at a proximal end and a screw engager at the distal end; and
a sleeve coupled to the elongate member;
translating the screw retention device away from the screw head with the sleeve;
gripping the screw head with the screw engager; and
axially translating the screw with the screw engager while the sleeve maintains the screw retention device away from the screw head.

28. The method as in claim 27 wherein translating the screw retention device comprises rotating the sleeve.

29. The method as in claim 27 wherein gripping the screw head comprises engaging a recess in the screw head with the screw engager, the screw engager comprising a split tip.

30. The method as in claim 29 wherein engaging the recess comprises inserting the split tip into the recess when the split tip is in a first position, and expanding the split tip to a second position to engage the recess.

31. The method as in claim 27 wherein the screw engager comprises a left-hand threaded tip, and wherein gripping the screw head comprises screwing the threaded tip into the screw head.

32. The method as in claim 31 wherein screwing the screw engager threaded tip into the screw head creates a threaded engagement between the screw head and tip.

33. The method as in claim 32 wherein the screw engager threaded tip is a self drilling threaded tip.

34. The method as in claim 27 wherein gripping the screw head comprises engaging a periphery of the screw head with the screw engager.

35. The method as in claim 34 wherein the screw engager comprises a lip to engage the screw head periphery.

36. The method as in claim 27 wherein the sleeve is rotatably and removably coupled to the elongate member.

37. The method as in claim 27 wherein the screw is stripped in a bone.

38. The method as in claim 37 wherein the bone is a vertebral body.
